(19) United States
(12) Patent Application Publication
(10) Pub. No.: US 2016/0015501 A1
(43) Pub. Date: Jan. 21, 2016

(54) MEDICAL DEVICE

(71) Applicant: TERUMO KABUSHIKI, Tokyo (JP)

(72) Inventors: Masakatsu KAWAURA, Sunnyvale, CA (US); Nao YOKOI, Sunnyvale, CA (US)

(73) Assignee: TERUMO KABUSHIKI, Tokyo (JP)

(21) Appl. No.: 14/870,181

(22) Filed: Sep. 30, 2015

Related U.S. Application Data
(63) Continuation of application No. PCT/JP2013/059888, filed on Apr. 1, 2013.

(51) Int. Cl. A61F 2/00; A61B 17/34; A61B 19/02

(52) U.S. Cl. CPC A61F 2/0045; A61B 19/0264; A61B 17/3439; A61B 17/3468; A61B 2019/0267

(57) ABSTRACT

A medical device and method are disclosed to be used when indwelling a belt-shaped flexible implant into a living body. The medical device includes: an elongated puncture needle which, by piercing the living body, forms a primary insertion hole in the living body; and a sheath into and out of which the puncture needle can be inserted and removed and which, by passing through the primary insertion hole in an assembled state with the puncture needle inserted therein, makes the primary insertion hole into a secondary insertion hole so as to permit the implant to be inserted in and passed through the secondary insertion hole. The puncture needle has a puncture needle-side expanding section which, when forming the primary insertion hole, expands the primary insertion hole to the same degree as the width of the implant.

### CROSS-REFERENCES TO RELATED APPLICATIONS

This application is a continuation of International Application No. PCT/JP2013/059888 filed on Apr. 1, 2013, the entire content of which is incorporated herein by reference.

### TECHNICAL FIELD

The present disclosure relates to a medical device.

### BACKGROUND DISCUSSION

If a person suffers from a urinary incontinence, for example, if a person suffers from a stress urinary incontinence, then urine leakage can be caused by application of abdominal pressure during normal exercise or by laughing, coughing, sneezing or the like. The cause of this may be, for example, that the pelvic floor muscle which is a muscle for supporting the urethra is loosened by birth or the like.

For the treatment of urinary incontinence, a surgical treatment is effective, in which there is used, for example, a tape-shaped implant called “sling.” The sling is indwelled inside the body and the urethra is supported by the sling (see, for example, Japanese Patent Laid-Open No. 2010-99499). In order to indwell the sling inside the body, an operator would incise the vagina with a surgical knife, dissect the biological tissue between the urethra and vagina, and, using a puncture needle or the like, make the dissected region and the outside communicate with each other, forming a puncture hole. Then, by use of such a puncture hole, the sling is indwelled into the body.

When dissecting the biological tissue, however, the degree of the dissection may be excessively high or may be insufficient. If the degree of dissection is excessively high, the sling can be instable upon being placed indwelling, and can therefore be unable to satisfactorily support the urethra. When the degree of dissection is insufficient, for example, the sling can be placed indwelling in a shrunk state; in this case, the sling can also be unable to satisfactorily support the urethra.

### SUMMARY

A medical device is disclosed by which an insertion hole having a required minimum size for indwelling of an implant in the medical device that can be formed relatively easily and reliably.

In accordance with an exemplary embodiment, a medical device is disclosed for indwelling a belt-shaped flexible implant into a living body, the medical device can include an elongated puncture needle which, by piercing the living body, forms a primary insertion hole in the living body and a sheath into and out of which the puncture needle can be inserted and removed and which, by passing through the primary insertion hole in an assembled state with the puncture needle inserted therein, makes the primary insertion hole into a secondary insertion hole so as to permit the implant to be inserted in and passed through the secondary insertion hole, wherein the puncture needle has a puncture needle-side expanding section which, when forming the primary insertion hole, expands the primary insertion hole to a same degree as a width of the implant.

In the medical device, preferably, the puncture needle can be formed, at least at part in a longitudinal direction of the puncture needle, with a flat section which is flat shaped in cross section, and the flat section functions as the puncture needle-side expanding section.

In the medical device, the sheath may have a sheath-side expanding section which, when forming the secondary insertion hole, can cause the secondary insertion hole to retain an expanded state of the primary insertion hole expanded by the puncture needle-side expanding section.

In another aspect, a medical device to be used when indwelling a belt-shaped flexible implant into a living body can include a puncture needle which, by piercing the living body, forms a primary insertion hole in the living body and a sheath into and out of which the puncture needle can be inserted and removed and which, by passing through the primary insertion hole in an assembled state with the puncture needle inserted therein, makes the primary insertion hole into a secondary insertion hole so as to permit the implant to be inserted in and passed through the secondary insertion hole, wherein the sheath has a sheath-side expanding section which, when forming the secondary insertion hole, expands the secondary insertion hole to a same degree as a width of the implant.

In the medical device, preferably, the sheath is formed, at least at part in a longitudinal direction of the sheath, with a flat section which is flat shaped in cross section, and the flat section functions as the sheath-side expanding section.

In the medical device, at least one of the puncture needle and the sheath may have a rotation restraining section which restrains rotation of the sheath relative to the puncture in the assembled state.

In the medical device, preferably, each of the puncture needle and the sheath is formed, at least at part in a longitudinal direction of the puncture needle and the sheath, with a flat section which is flat shaped in cross section, and the flat sections, by overlapping with each other in the assembled state, function as the rotation restraining section.

In the medical device, an overall length of the sheath may be greater than an overall length of the puncture needle.

In the medical device, preferably, the puncture needle has a curved shape by being curved in a circular arc shape.

In the medical device, for example, preferably, the sheath is flexible and can be, in the assembled state, deformed so as to be along the curved shape.

In the medical device, the sheath may be preliminarily re-shaped so as to be along the curved shape in the assembled state.

The medical device may further include an elongated insertion section to be inserted into a biological lumen, wherein the sheath is formed, at least at part in a longitudinal direction of the sheath, with a flat section which is flat shaped in cross section, and the flat section can, when the sheath forms the secondary insertion hole, be disposed in parallel to the insertion section.

The medical device may be used for treating a disorder in a pelvic organ by indwelling the implant between a urethral lumen and a vaginal cavity.

The medical device may include the implant and an implant package having a wrapping material, the wrapping material including a bag-shaped wrapping material main body in which to accommodate the implant, and a flexible linear body for pulling the wrapping material main body.

In the medical device, preferably, the implant has a stopper, which makes contact with a body surface when the implant is indwelled in the living body.

According to the described aspects and above-mentioned configurations, the formation of the primary insertion hole by the puncture needle or the formation of the secondary insertion hole by the sheath can be performed relatively easily and reliably. Both of the primary insertion hole and the secondary insertion hole have been expanded to the same degree as the width of the implant, and they have a necessary and sufficient size for stable indwelling of the implant therein, namely, a required minimum size for the stable indwelling. When the implant is inserted in and passed through the insertion hole formed in this way, the implant is prevented from shrinking in the width direction, is put in the state of being developed (unfolded) sufficiently, and is left indwelling stably.

For example, where the medical device of the present disclosure is used for treatment of female urinary incontinence, the urethra can be satisfactorily supported from the vagina side by the implant inserted in and passed through the insertion hole. Consequently, the treatment of female urinary incontinence can be achieved reliably.

A medical device is disclosed for indwelling a belt-shaped flexible implant into a living body, the medical device comprising: an elongated puncture needle which, by piercing the living body, forms a primary insertion hole in the living body; and a sheath into and out of which the puncture needle can be inserted and removed and which, by passing through the primary insertion hole in an assembled state with the puncture needle inserted therein, makes the primary insertion hole into a secondary insertion hole so as to permit the implant to be inserted in and passed through the secondary insertion hole, wherein the puncture needle has a puncture needle-side expanding section which, when forming the primary insertion hole, expands the primary insertion hole to a same degree as a width of the implant.

A medical device is disclosed for indwelling a belt-shaped flexible implant into a living body, the medical device comprising: a puncture needle which, by piercing the living body, forms a primary insertion hole in the living body; and a sheath into and out of which the puncture needle can be inserted and removed and which, by passing through the primary insertion hole in an assembled state with the puncture needle inserted therein, makes the primary insertion hole into a secondary insertion hole so as to permit the implant to be inserted in and passed through the secondary insertion hole, wherein the sheath has a sheath-side expanding section which, when forming the secondary insertion hole, expands the secondary insertion hole to a same degree as a width of the implant.

A method is disclosed of indwelling a belt-shaped flexible implant into a living body, the method comprising: inserting a medical device into the living body, the medical device including an elongated puncture needle which, by piercing the living body, forms a primary insertion hole in the living body, and a sheath into and out of which the puncture needle can be inserted and removed; passing the medical device through the primary insertion hole in an assembled state with the puncture needle inserted in the sheath; making the primary insertion hole into a secondary insertion hole so as to permit the implant to be inserted in and passed through the secondary insertion hole; and wherein the puncture needle has a puncture needle-side expanding section which, when forming the primary insertion hole, expands the primary insertion hole to a same degree as a width of the implant.

### BRIEF DESCRIPTION OF THE DRAWINGS

FIG. 1 is a side view showing a medical device according to an illustrative embodiment of the present disclosure;

FIG. 2 is a sectional view taken along line 2-2 of FIG. 1;

FIG. 3 is a view (as viewed from the direction of arrow A in FIG. 1) for explaining an operating procedure of the medical device shown in FIG. 1;

FIG. 4 is a view (as viewed from the direction of arrow A in FIG. 1) for explaining the operating procedure of the medical device shown in FIG. 1;

FIG. 5 is a view (as viewed from the direction of arrow A in FIG. 1) for explaining the operating procedure of the medical device shown in FIG. 1;

FIG. 6 is a view (as viewed from the direction of arrow A in FIG. 1) for explaining the operating procedure of the medical device shown in FIG. 1;

FIG. 7 is a view (as viewed from the direction of arrow A in FIG. 1) for explaining the operating procedure of the medical device shown in FIG. 1;

FIG. 8 is a view (as viewed from the direction of arrow A in FIG. 1) for explaining the operating procedure of the medical device shown in FIG. 1;

FIG. 9 is a view (as viewed from the direction of arrow A in FIG. 1) for explaining the operating procedure of the medical device shown in FIG. 1;

FIGS. 10A to 10C are views (showing a process of formation of a puncture hole by a puncture needle) as viewed from the direction of arrow A in FIG. 4;

FIG. 11 is a sectional view taken along line 11-11 of FIG. 5; and

FIG. 12 is a sectional view taken along line 12-12 of FIG. 9.

### DETAILED DESCRIPTION

A medical device according to the described aspects of the present disclosure will be described in detail below, referring to an illustrative preferred embodiment shown in the attached drawings.

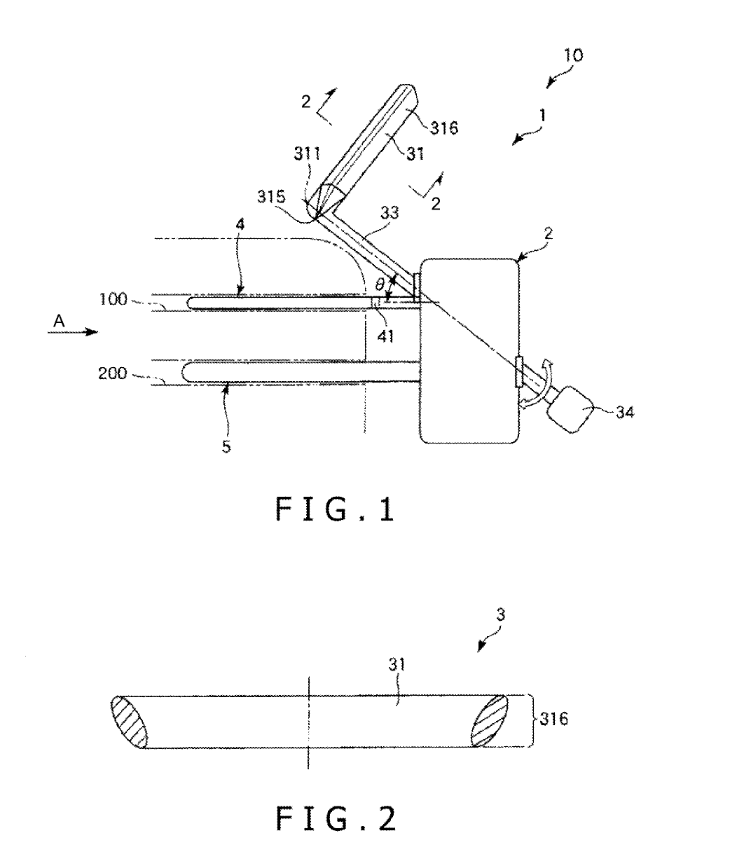

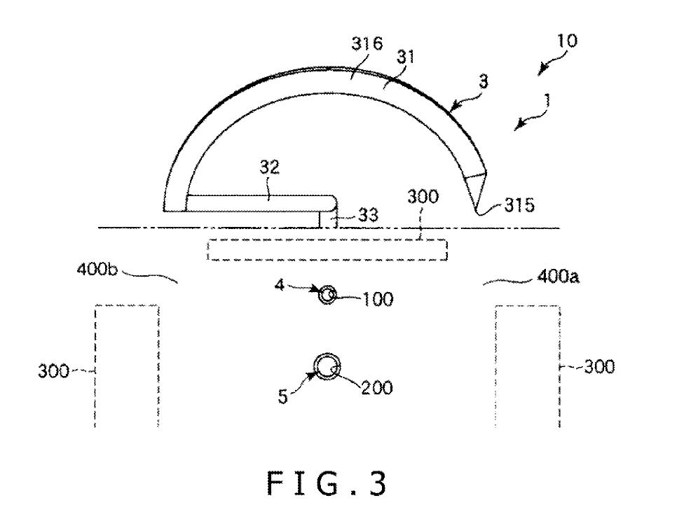

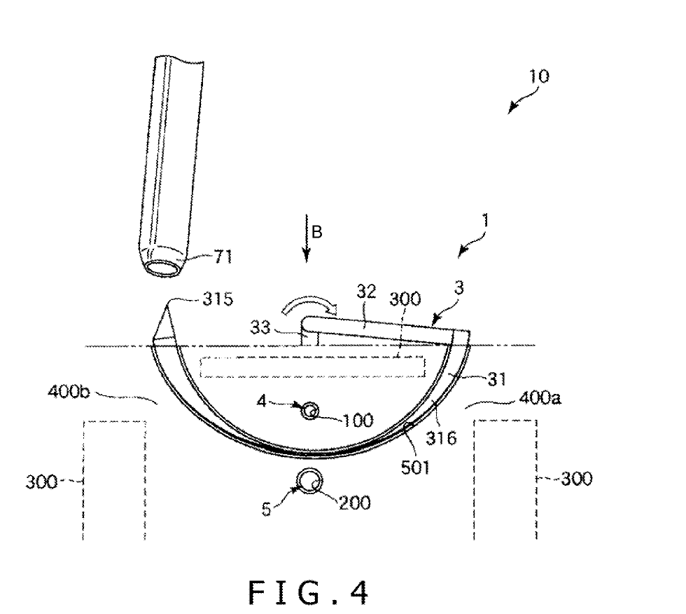

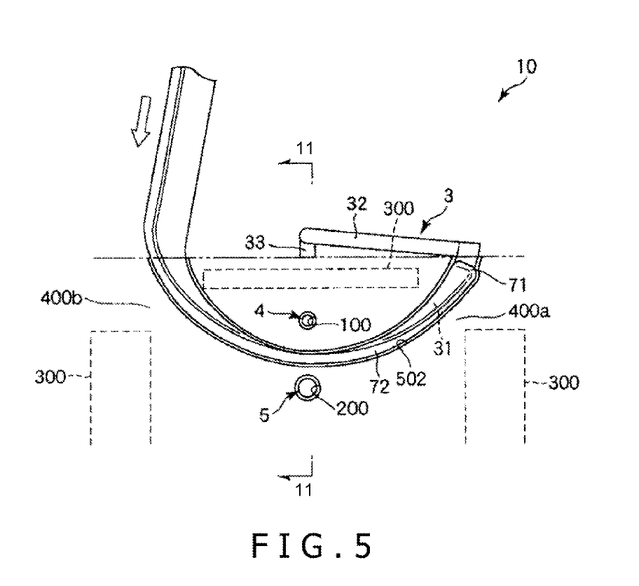

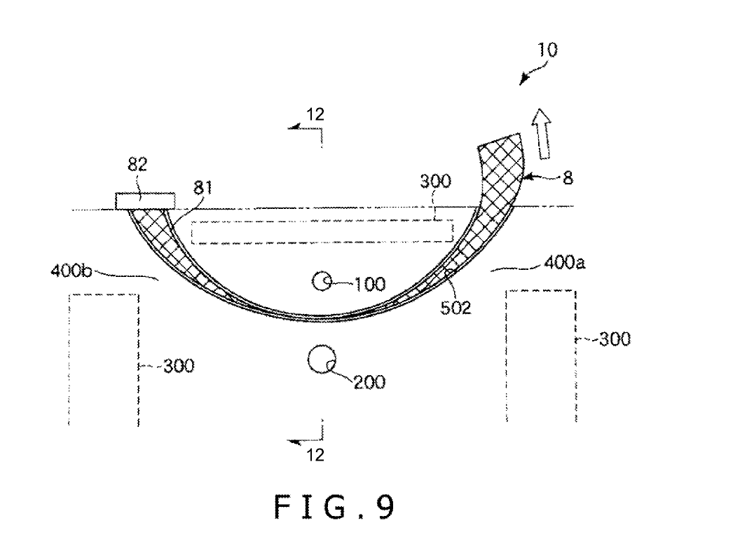

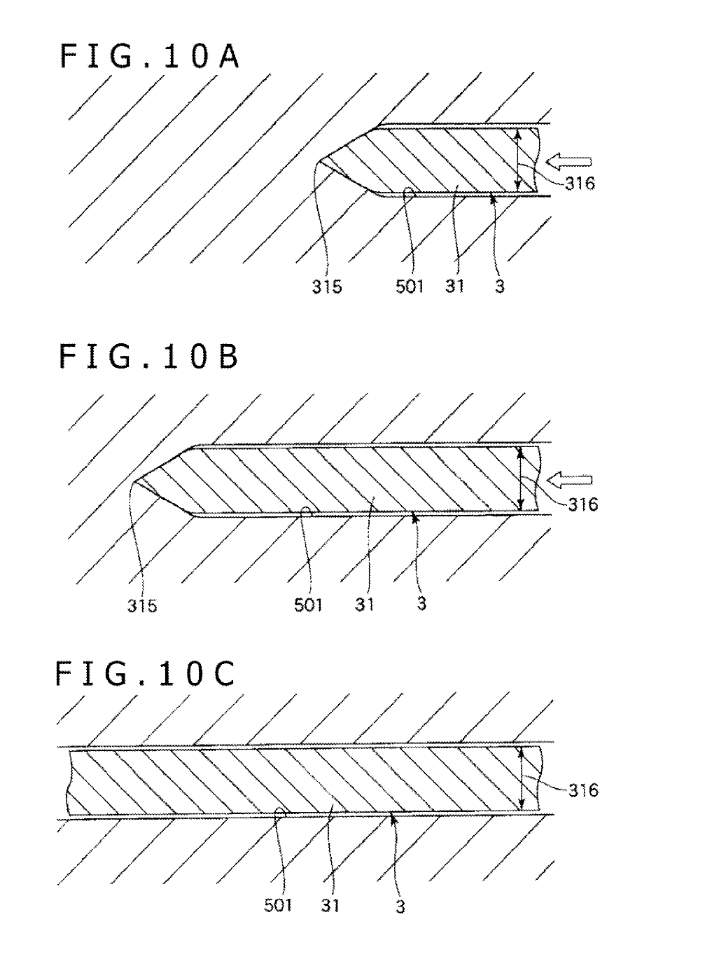

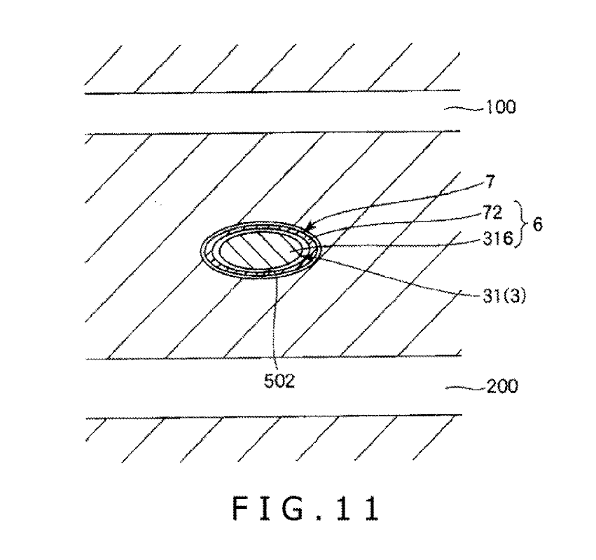

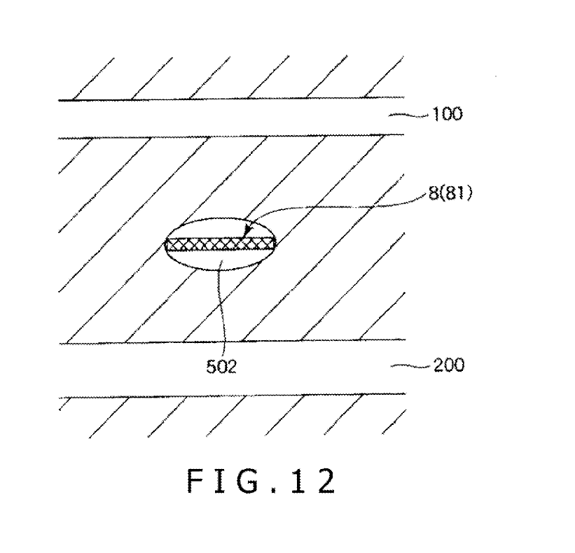

FIG. 1 is a side view showing a medical device according to an illustrative embodiment of the present disclosure; FIG. 2 is a sectional view taken along line 2-2 of FIG. 1; FIGS. 3 to 9 are views (as viewed from the direction of arrow A in FIG. 1) for explaining an operating procedure of the medical device shown in FIG. 1; FIGS. 10A to 10C are views (showing a process of formation of a puncture hole by a puncture needle) as viewed from the direction of arrow B in FIG. 4; FIG. 11 is a sectional view taken along line 11-11 of FIG. 5; and FIG. 12 is a sectional view taken along line 12-12 of FIG. 9. In the following, the needle tip side of a puncture needle will be referred to as “distal” side, and the opposite side as “proximal” side. Besides, the upper side in FIGS. 1, 3 to 9, 11, and 12 will be referred to as “upper (side),” and the lower side as “lower (side).”

A medical device 10 shown in FIGS. 1 and 3 to 9 is for use in treatment of a disorder in a pelvic organ, specifically, treatment of female urinary incontinence. In this treatment, an implant (in-body indwelling instrument) 8 can be indwelled (embedded or implanted) between a urethra (urethral lumen) 100 and a vagina (vaginal cavity) 200. The medical device 10 can include a puncture device 1 and a sheath 7. The configuration of each component will be described later.

An implant 8 is an embeddable instrument which is called “sling” and which can be used for treatment of female urinary incontinence, for example, for supporting the urethra 100. For example, the implant 8 is an instrument, which when the urethra 100 is going to move toward the vagina 200 side, supports the urethra 100 so as to restrict its movement in the direction for coming away from the vagina 200. As shown in FIG. 9, the implant 8 can include a belt-shaped flexible implant main body 81, and a stopper 82 fixed to a distal portion (one end portion) of the implant main body 81.

The implant main body 81 is not particularly limited. For example, the implant main body 81 may be composed of a network (lattice)-formed knitted body knitted by causing linear elements to intersect, specifically, a network-formed braiding. Examples of the linear element include those, which are circular in cross section, and those, which are flat shaped in cross section, namely, belt-shaped (ribbon-shaped) ones. The material constituting the implant main body 81 is not specifically restricted. Examples of the material usable here include various resin materials, which are biocompatible. Note that the implant main body 81 is naturally not limited to the network-formed one.

The stopper 82 is configured of a rigid member, which is greater than the implant main body 81 in width. The stopper 82 is a member, which makes contact with a body surface in a condition where the implant 8 is indwelled in a living body, which can help prevent the implant 8 from being drawn out of the living body even if the implant 8 is pulled toward the proximal side. The material constituting the stopper 82 is not particularly limited. Examples of the material applicable here include various resin materials. Note that the method for fixing the stopper 82 to the implant main body 81 is not specifically restricted, and, there can be used, for example, a method of fixing by welding (heat welding, high-frequency welding, ultrasonic welding or the like), and a method of fixing by adhesion (adhesion by use of an adhesive or a solvent).

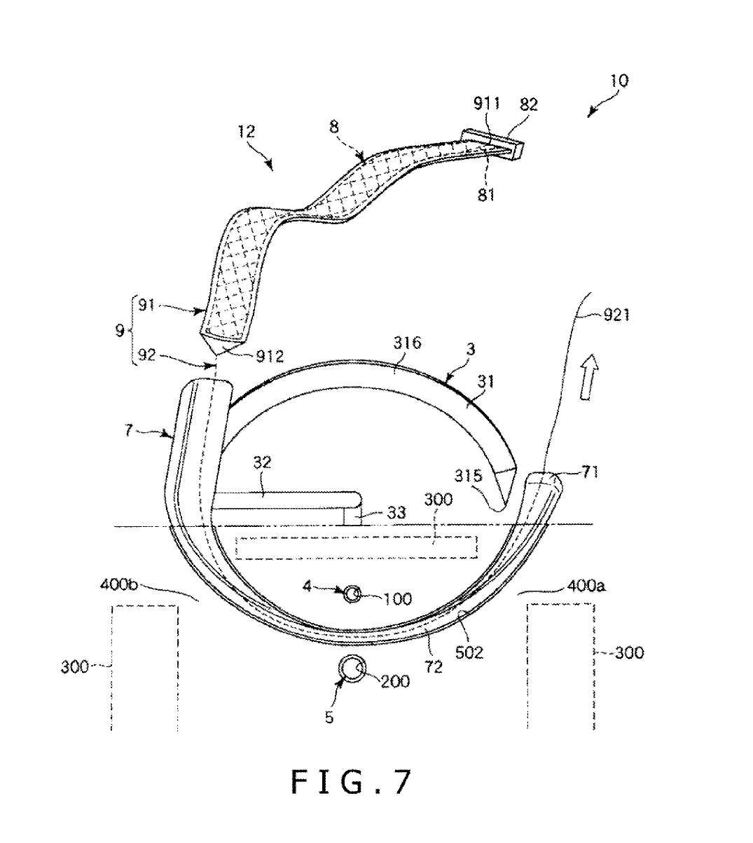

As depicted in FIG. 7, the implant 8 has the implant main body 81 accommodated in a wrapping material 9. The wrapping material 9 can include a wrapping material main body 91, which is elongated and flexible, and a string 92 fixed to a proximal portion 912 of the wrapping material main body 91.

The wrapping material main body 91 is a bag-shaped member, which is open at a distal portion 911 of the main body 91 and is closed at a proximal portion 912 of the main body 91. The wrapping material main body 91 has a length greater than the length of the implant main body 81, and a width greater than the width of the implant main body 81, which can help ensure that the implant main body 81 is prevented from shrinking inside the wrapping material main body 91 and remains in a sufficiently developed (unfolded) state. The width of the proximal portion 912 of the wrapping material main body 91 gradually decreases along the proximal direction. The material constituting the wrapping material main body 91 is not particularly limited, and, for example, various resin materials can be used as the material.

In the medical device 10, an implant package 12 is preliminarily prepared in which the implant 8 (implant main body 81) is accommodated in the wrapping material 9 (wrapping material main body 91). The implant package 12 can be, when inserted into the sheath 7, pulled by the string 92 fixed to the wrapping material main body 91. Note that the string 92 is longer than the overall length of the sheath 7.

As shown in FIGS. 1 and 3, the puncture device 1 can includes a puncture member 3 including a puncture needle 31 adapted to puncture a living body (biological tissue), a shaft section 33, and an interlock section 32 interlocking the puncture needle 31 and the shaft section 33, an elongated urethral-insertion member 4 to be inserted into the urethra 100, an elongated vaginal-insertion member 5 to be inserted into the vagina 200, and a support member (restraining means) 2 supporting the puncture member 3, the urethral-insertion member 4, and the vaginal-insertion member 5.

The urethral-insertion member 4, in this exemplary embodiment, can be supported by the support member 2 in a cantilever form. The urethral-insertion member 4 can be a straight hollow or solid body formed from a non-flexible rigid material. Note that in the case where the urethral-insertion member 4 is a hollow body, an end portion on the free end side of the urethral-insertion member 4 is preferably open. In this case, a balloon catheter (not illustrated) having an expandable and contractible balloon can be inserted in the urethral-insertion member 4. Then, in the condition where the balloon is protruded inside a patient's bladder, the balloon can be expanded. The thus expanded balloon is caught on a bladder neck, whereby the position of the urethral-insertion member 4 relative to the bladder and the urethra 100 can be fixed.

As depicted in FIG. 1, a marker 41 can be provided on a peripheral portion of the urethral-insertion member 4. The marker 41 can be so disposed that the marker 41 is positioned at a urethral opening at the time when the urethral-insertion member 4 is inserted in the urethra 100 and the end portion of the urethral-insertion member 4 is positioned immediately on the operator's side of the bladder.

The vaginal-insertion member 5, in this exemplary embodiment, can be supported by the support member 2 in a cantilever form. The vaginal-insertion member 5 is straight bar-like in shape. In accordance with an exemplary embodiment, an end portion on the free end side of the vaginal-insertion member 5 can be rounded, which can help enable a smooth insertion of the vaginal-insertion member 5 into the vagina.

The vaginal-insertion member 5 is disposed on the lower side of the urethral-insertion member 4, at a predetermined distance from the urethral-insertion member 4, so that its axis is parallel to the axis of the urethral-insertion member 4.

Note that the materials constituting the urethral-insertion member 4, the vaginal-insertion member 5, and the support member 2 are not particularly limited, and, for example, various resin materials and the like can be used for these members.

The puncture member 3 has its shaft section 33 (which serves as a rotating shaft of the puncture member 3) turnably disposed on the support member 2.

The shaft section 33 penetrates the support member 2, and is inhibited from moving in the axial direction relative to the support member 2.

As shown in FIG. 1, the shaft section 33 is inclined against the axis of the urethral-insertion member 4 so that the distance between the axis of the shaft section 33 and the axis of the urethral-insertion member 4 increases toward the left side in FIG. 1. The inclination angle θ can be, for example, preferably 20 to 60 degrees, more preferably 30 to 45 degrees, and further preferably 35 to 40 degrees, which can help ensure that puncture by the puncture needle 31 can be carried out relatively easily, and a shorter puncture distance can be adopted.

For example, with the inclination angle θ set within the above-mentioned range, the puncture needle 31 can capture the left and right obturator foramens 400a and 400b of the pelvis wider on a planar basis, and a wider puncture space for the puncture needle 31 can be secured. In accordance with an exemplary embodiment, for example, in a condition where the patient is set in a predetermined position (lithotomy position), the puncture needle 31 can be made to puncture the biological tissue in a direction comparatively nearer to a perpendicular direction relative to the left and right obturator foramens 400a and 400b of the pelvis. Therefore, the puncture by the puncture needle 31 can be performed relatively easily. In addition, with the puncture needle 31 made to puncture the biological tissue in a direction comparatively nearer to a perpendicular direction relative to the obturator foramens 400a and 400b, the puncture needle 31 passes a shallow portion of tissue, so that a needle tip 315 of the puncture needle 31 can pass between the left and right obturator foramens 400a and 400b while taking the course of a shorter distance. Since the puncture needle 31 is made to pass those zones in the obturator foramens 400a and 400b, which are comparatively nearer to the pubic symphysis, preferably safety zones, it is possible to puncture a region safety where there are few nerves and blood vessels, which should be prevented from being damaged. Consequently, the puncturing procedure is less invasive, and a lighter burden on the patient can be realized. Thus, with the inclination angle θ set within the above-mentioned range, the puncture of the patient by the puncture needle 31 can be performed suitably. If the inclination angle θ is below the above-mentioned lower limit or above the above-mentioned upper limit, on the other hand, there may arise a situation, depending on the individual differences concerning the patient or the posture of the patient during the procedure or the like, in which the puncture needle 31 cannot capture the obturator foramens 400a and 400b wide on a planar basis or in which a sufficiently short puncture route cannot be realized. Therefore, it can be preferable to make the puncture needle 31 puncture the biological tissue perpendicularly in relation to the left and right obturator foramens 400a and 400b of the pelvis.

By puncturing at the aforementioned angle, it can be made easy to aim at a tissue present between a middle-part urethra (which refers to a middle part in the longitudinal direction of the urethra 100) and the vagina 200. The region between the middle-part urethra and the vagina 200 is a position suitable for treatment of urinary incontinence by embedding the implant 8 in the region between the middle-part urethra and the vagina 200. More preferably, puncturing is conducted in a condition where the urethra 100 or the vagina 200 or both of the urethra 100 and the vagina 200 have been positionally shifted in the manner of being pushed toward the inner side of the body, whereby it is ensured that puncturing between the middle-part urethra and the vagina 200 is achieved easily. Means for pushing either one of the urethra 100 and the vagina 200 toward the inner side of the body may be, for example, as follows. After the urethral-insertion member 4 and/or the vaginal-insertion member 5 has been inserted into an appropriate position, and before puncturing, the urethral-insertion member 4 and/or the vaginal-insertion member 5 is moved toward the inner side of the body along its axis to a predetermined position. In this case, if the urethral-insertion member 4 and/or the vaginal-insertion member 5 is provided with a visible marker or a marker recognizable on a non-invasive intracorporeal-image monitor based on X-rays, ultrasound or the like, the distance the relevant member is moved can be recognized.

In a condition where at least one of the urethra 100 and the vagina 200 is positionally shifted in the manner of being pushed toward the inner side of the body, the puncture needle 31 is made to puncture the biological tissue perpendicularly in relation to the left and right obturator foramens 400a and 400b of the pelvis, whereby a passage can be formed in a position suitable for indwelling the implant 8 therein.

It can be preferable to adopt a setting such that the trajectory of the puncture needle 31 passes the safety zones in the left and right obturator foramens 400a and 400b of the pelvis, to positionally shift at least one of the urethra 100 and the vagina 200 so that the trajectory is located between the middle-part urethra and the vagina 200, and to make the puncture needle 31 puncture along the trajectory, thereby forming a passage.

The puncture needle 31 can have the sharp needle tip 315 at the distal end of the puncture needle 31, and can have a curved shape of being curved in a circular arc shape centered on the shaft section 33. The puncture needle 31 can be inserted into and removed out of the sheath 7 (see FIGS. 3 to 6). Note that the center angle of the circular arc of the puncture needle 31 is not particularly limited; for example, the center angle is preferably 150 to 270 degrees, more preferably 170 to 250 degrees, and further preferably 190 to 230 degrees.

In FIG. 1, the axis of the puncture needle 31 and the axis of the shaft section 33 are in a positional relation of skew lines, which can help ensure that when the puncture needle 31 is turned, the needle tip 315 of the puncture needle 31 moves in a plane orthogonal to the axis of the shaft portion 33, namely, a plane to which the axis is normal, along the circular arc.

While the needle tip 315 of the puncture needle 31 is oriented clockwise in the figures in this embodiment, this is not restrictive, and the needle tip 315 may be oriented counterclockwise in the figures.

The puncture needle 31 may be a solid needle or a hollow needle.

In this embodiment, the puncture needle 31 is disposed at a position, in the axial direction of the urethral-insertion member 4, corresponding to an intermediate part in the longitudinal direction of the urethral-insertion member 4.

In accordance with an exemplary embodiment, for example, the supporting member 2 can regulate the positional relation of the puncture needle 3 and the urethral-insertion member 4 so that when the puncture member 3 is turned to puncture a living body, the needle tip 315 of the puncture needle 31 passes the farther side from a center 311 of the puncture needle 31 than the urethral-insertion member 4 or an extension line thereof, namely, the lower side of the urethral-insertion member 4 or the extension line thereof. Note that the center 311 of the puncture needle 31 is the center of the circular arc of the puncture needle 31, namely, the center of turning of the puncture needle 31 (puncture member 3).

In addition, the support member 2 can regulate the positional relation of the puncture member 3 and the vaginal-insertion member 5 so that the needle tip 315 of the puncture needle 31 does not collide on the vaginal-insertion member 5 or the extension line thereof when the puncture needle 3 is turned to puncture the living body.

For example, the support member 2 can regulate the positional relation of the puncture member 3 and the urethral-insertion member 4 and the vaginal-insertion member 5 so that when the puncture needle 3 is turned to puncture the biological tissue, the needle tip 315 of the puncture needle 31 passes between the urethral-insertion member 4 or the extension line thereof and the vaginal-insertion member 5 or the extension line thereof, which can help ensure that the living body can be punctured by the puncture needle 31 while avoiding the urethra 100 and the vagina 200. Consequently, puncture of the urethra 100 by the puncture needle 31 and puncture of the vagina 200 by the puncture needle 31 are both prevented.

In addition, for example, since the trajectory of the needle tip 315 of the puncture needle 31 can be definite, the operator himself/herself can be prevented from puncturing his/her finger with the puncture needle 31, which can help ensure safety.

The center angle of the circular arc of the puncture needle 31 is not particularly limited, and may be appropriately set according to various conditions. However, the center angle is so set that when puncturing the living body by the puncture needle 31, the puncture needle 31 can enter the living body via the body surface on one side of the patient, pass on the lower side of the urethra, and go out of the body via the body surface on the other side. In accordance with an exemplary embodiment, for example, the center angle concerning the puncture needle 31 is preferably 120 to 270 degrees, more preferably 160 to 230 degrees, and further preferably 180 to 210 degrees, which can help ensure that when puncturing the living body by the puncture needle 31, the puncture needle 31 can reliably enter the living body via the body surface on one side of the patient, pass on the lower side of the urethra 100, and go out of the body via the body surface on the other side (see FIG. 4).

At a proximal portion of the shaft section 33, there is provided a grip section 34 as an operating section for turning the puncture member 3. The grip section 34 is rectangular parallelepiped in shape in this exemplary embodiment. At the time of turning the puncture member 3, the grip section 34 is gripped with fingers and is turned in a predetermined direction. Note that the shape of the grip section 34 is naturally not restricted to the just-mentioned.

Note that the material constituting the puncture needle 3 is not specifically restricted, and examples of the material which can be used include various metallic materials such as stainless steel, aluminum or aluminum alloys, titanium or titanium alloys, etc.

In accordance with an exemplary embodiment, the medical device 10 can assume a first state in which the puncture needle 31 of the puncture member 3 is not yet inserted in the sheath 7 or has been drawn out of the sheath 7 as depicted in FIGS. 1, 3, 4, 6, and 7, and a second state (assembled state) in which the puncture needle 31 has been inserted into the sheath 7 to complete assembly as shown in FIG. 5.

As shown in FIG. 4, in the first state, a living body can be punctured by the puncture needle 31 present as a single element. By this, a primary insertion hole 501 can be formed in the living body. As shown in FIG. 5, in the second state, the primary insertion hole 501 can be made into a second insertion hole 502 by the passage of the sheath 7 through the primary insertion hole 501. The primary insertion hole 501 and the second insertion hole 502 are both penetrating holes (through-holes) in and through which the implant 8 can be inserted and passed.

As shown in FIG. 2, the puncture needle 31 is flat shaped in cross section. The flat section thus flat shaped in cross section is formed over the whole part in the longitudinal direction of the puncture needle 31, namely, over the full length of the puncture needle 31. The flat section functions as a puncture needle-side expanding section 316 which, when the puncture needle 31 forms the primary insertion hole 501, expands the primary insertion hole 501 to the same degree as the width of the implant main body 81 of the implant 8.

Note that as shown in FIGS. 10A to 10C, the expanding direction in expanding to the same degree as the width of the implant main body 81 is principally the vertical direction in the figures, namely, a direction inclined by the inclination angle θ against the center axis of the curved shape (of being curved into a circular arc shape) of the puncture needle 31.

The above-mentioned flat shape is not particularly limited. For example, the flat shape is preferably an ellipse as depicted in FIG. 2, and can be a rhombic, a UFO-like shape, or other flat shape.

At a portion (distal portion) near the needle tip 315 of the puncture needle 31, the width of the puncture needle-side expanding section 316 gradually decreases along the distal direction, and, preferably, the thickness of the puncture needle-side expanding section 316 also gradually decreases along the distal direction.

The sheath 7 is configured of a flexible tubular body. The sheath 7, in the second state, can be deformed to be along, or to follow, the curved shape (of being curved into a circular arc shape) of the puncture needle 31. Alternatively, the sheath 7 may be preliminarily re-shaped so as to be along the above-mentioned curved shape in the assembled state. Note that the material constituting the sheath 7 is not particularly limited, and, for example, various resin materials can be used as the material.

As shown in FIG. 5, the overall length of the sheath 7 is greater than the overall length of the puncture needle 31, which helps ensure that when the sheath 7 is pushed in toward the puncture needle 31 in the state of puncturing the living body, the sheath 7 can be reliably pushed in until passed through the primary insertion hole 501. In addition, there is a merit that a mesh (implant 8) can be preliminarily disposed in the sheath 7.

A proximal portion 71 of the sheath 7 is tapered so that its outside diameter gradually decreases along the proximal direction, which helps ensure that when the sheath 7 is pushed into the primary insertion hole 501, starting from the proximal portion 71 side, as shown in FIGS. 4 and 5, the pushing-in operation can be carried out relatively easily

As shown in FIG. 11, the sheath 7 is flat shaped in cross section, like the puncture needle 31. The flat section thus flat shaped functions as a sheath-side expanding section 72 which, when the sheath 7 forms the secondary insertion hole 502, helps permit the secondary insertion hole 502 to securely retain the expanded state of the primary insertion hole 501 expanded by the puncture needle-side expanding section 316, for example, to be reliably expanded to the same degree as the width of the implant main body 81 of the implant 8.

When the implant main body 81 of the implant 8 is inserted into and passed through the secondary insertion hole 502 thus formed, the implant main body 81 can be prevented from shrinking in the width direction of the implant body 81, is put in a sufficiently developed (unfolded) state, and is placed indwelling stably (see FIG. 12). As a result, the urethra 100 can be satisfactorily supported from the vagina 200 side, and, accordingly, the treatment of female urinary incontinence can be reliably performed.

In the second state, the puncture needle-side expanding section 316 of the puncture needle 31 that is flat shaped in cross section and the sheath-side expanding section 72 of the sheath 7 that is flat shaped in cross section overlap with each other (see FIG. 11). Thus, in the medical device 10, the parts that are flat shaped in cross section overlap with each other, whereby a function as a rotation restraining section 6 for restraining rotation of the sheath 7 is exhibited. By the rotation restraining section 6, the sheath 7 is restrained in rotation about its center axis in relation to the puncture needle 31. As a result, the expanding direction in the secondary insertion hole 502 is also in the same direction as the expanding direction of the primary insertion hole 501. Accordingly, the secondary insertion hole 502 such that the implant main body 81 can be sufficiently developed (unfolded) therein can be formed reliably.

A method is disclosed of using the medical device 10, for example, a procedure of embedding the implant 8 into a living body, will be described below referring to FIGS. 1 and 3 to 9.

First, as shown in FIGS. 1 and 3, the puncture device 1 of the medical device 10 is mounted onto a patient. Specifically, the urethral-insertion member 4 of the puncture device 1 is inserted into the patient's urethra 100, and the vaginal-insertion member 5 is inserted into the patient's vagina 200. In this instance, it can be relatively ensured that the marker 41 is positioned at the urethral opening or just on the operator's side of the urethral opening. By this, an end portion of the urethral-insertion member 4 can be positioned just on the operator's side of the bladder.

Next, as shown in FIG. 4, the grip section 34 of the puncture member 3 is gripped, and the puncture member 3 is rotated clockwise in the figure. By this, the needle tip 315 of the puncture needle 31 is moved clockwise in FIG. 4 along the circular arc thereof, to puncture the patient body surface at an inguinal region on the right side in the figure or near the inguinal region, enter the body, pass the obturator foramen 400a of a pelvis 300, pass on the lower side of the urethra 100, specifically, pass between the urethra 100 and the vagina 200, pass the obturator foramen 400b of the pelvis 300, and protrudes to the exterior of the body via the body surface at an inguinal region on the left side in the figure or near this inguinal region. As a result, the patient is formed therein with a primary insertion hole 501 which extends from the body surface at the inguinal region on the right side in FIG. 4 or near the inguinal region, extends through the obturator foramen 400a, then between the urethra 100 and the vagina 200, and through the obturator foramen 400b, to reach the body surface at the inguinal region on the left side in the figure or near this inguinal region. As aforementioned, the primary insertion hole 501 is a penetrating hole expanded to the same degree as the width of the implant main body 81 of the implant 8.

Subsequently, the sheath 7 of the medical device 10 is prepared, and, as shown in FIG. 5, the sheath 7 is pushed into the primary insertion hole 501 starting from the proximal end of the sheath 7, while keeping the sheath 7 along the puncture needle 31 in the state of being inserted in and passed through the primary insertion hole 501. As a result, the sheath 7 is passed through the primary insertion hole 501 while coming into the second state. By this passage, the primary insertion hole 501 is made into the secondary insertion hole 502. As aforementioned, the secondary insertion hole 502 is a penetrating hole in which the expanded state of the primary insertion hole 501 is securely maintained.

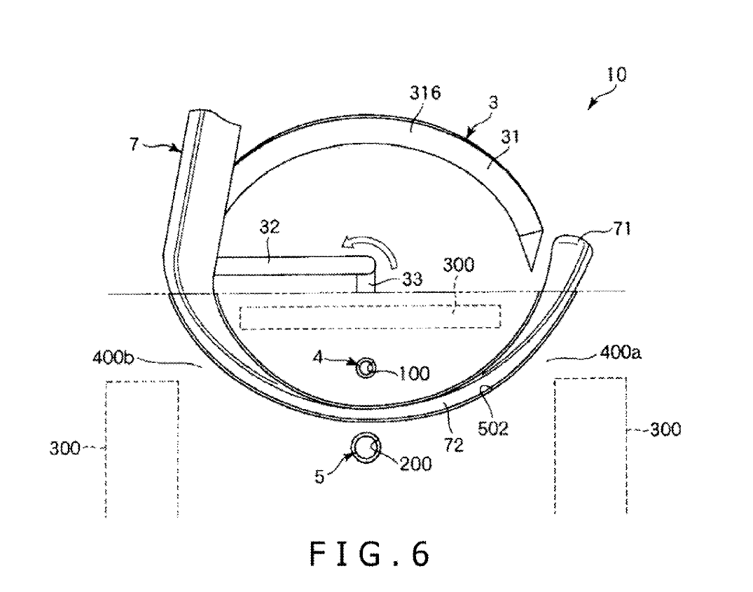

Next, as shown in FIG. 6, the grip section 34 of the puncture member 3 is gripped, and the puncture member 3 is rotated counterclockwise in the figure, with the sheath 7 left in the secondary insertion hole 502, which causes the needle tip 315 of the puncture needle 31 to move counterclockwise in FIG. 6 along the circular arc thereof, enter the body via the body surface at the inguinal region on the left side in the figure of the patient or near the inguinal region, pass the obturator foramen 400b of the pelvis 300, pass on the lower side of the urethra 100, for example, between the urethra 100 and the vaginal 200, pass the obturator foramen 400a of the pelvis 300, and comes out of the body via the body surface at the inguinal region on the right side in the figure or near this inguinal region. For example, the puncture needle 31 is drawn out of the body.

Subsequently, as shown in FIG. 7, the string 92 of the implant package 12 is inserted into and passed through the sheath 7. As a result, a proximal portion 921 of the string 92 protrudes from the proximal end of the sheath 7.

Then, the proximal portion 921 of the string 92 thus protruding is gripped, and the string 92 is pulled. As a result, the implant 8 can be inserted into and passed through the sheath 7 together with the wrapping material 9.

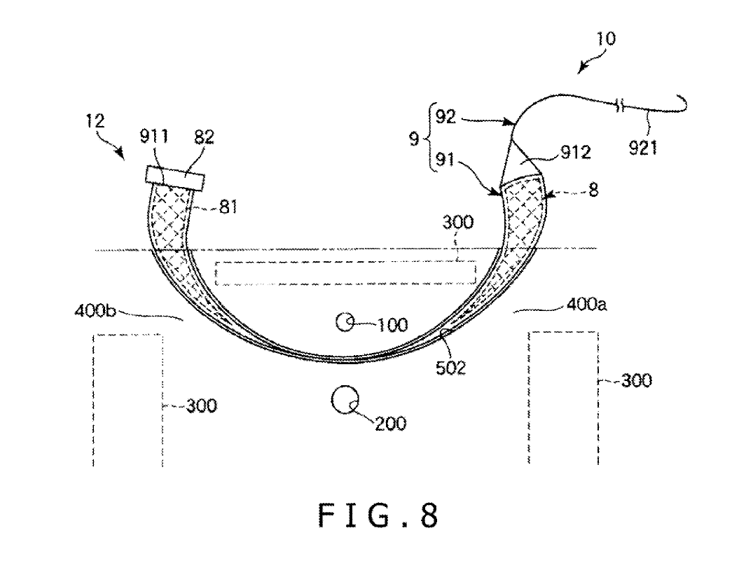

Next, as shown in FIG. 8, the proximal portion 71 of the sheath 7 is gripped, and the sheath 7 is pulled, with the implant package 12 left in the secondary insertion hole 502. As a result, the sheath 7 can be drawn out of the secondary insertion hole 502.

In addition, the puncture device 1 can be dismounted from the patient. For example, the urethral-insertion member 4 can be drawn out of the urethra 100, and the vaginal-insertion member 5 can be drawn out of the vagina 200 of the patient.

Subsequently, as shown in FIG. 9, the whole part of the implant package 12 is drawn until the stopper 82 makes contact with the body surface, followed by pulling the wrapping material 9, with the implant 8 left in the secondary insertion hole 502. As a result, the wrapping material 9 can be drawn out of the secondary insertion hole 502, and the implant 8 can be left indwelling in the secondary insertion hole 502. In this instance, the implant main body 81 of the implant 8 is in the state of being sufficiently developed (unfolded) in the width direction. For example, both sides of the implant main body 81 respective face to the urethra 100 side and to the vagina 200 side (see FIG. 12).

Then, the implant 8 is pulled with predetermined forces, to adjust the position of the implant 8 relative to the urethra 100, unnecessary portions of the implant main body 81 are cut away, and a predetermined treatment or treatments are conducted, to finish the procedure. As a result, the implant 8 is stably indwelled in the secondary insertion hole 502, so that the implant 8 can support the urethra 100 from the vagina 200 side in a satisfactory manner. Accordingly, the treatment of female urinary incontinence can be reliably performed.

Thus, according to the medical device 10, before indwelling of the implant 8, the insertion hole (primary insertion hole 501 and secondary insertion hole 502) which is flat shaped in cross section like the implant 8 can be formed relatively easily and reliably. In accordance with an exemplary embodiment, for example, the insertion hole has a required minimum size for stable indwelling of the implant 8.

In addition, according to the medical device 10, the surgical incision and dissection at the time of indwelling of an implant, as conducted in the conventional urinary incontinence treatment, can be omitted. By this, the burden on the patient can be reduced, and, hence, low invasive treatment of urinary incontinence can be realized. In accordance with an exemplary embodiment, for example, the safety of the patient can be relatively high, and the safety of the operator can also be relatively high.

Further, the living body can be punctured by the puncture needle 31 while avoiding the urethra 100 and the vagina 200, so that the puncture needle 31 can be prevented from puncturing the urethra 100 or the vagina 200. Thus, safety can be relatively ensured. In accordance with an exemplary embodiment, for example, the operator himself/herself can be prevented from puncturing his/her finger with the puncture needle 31, so that safety can be relatively ensured.

While the medical device of the present disclosure has been described above with reference to the embodiment illustrated in the attached drawings, the disclosure is not limited to the embodiment. Each component of the medical device can be replaced with arbitrarily configured one that can exhibit the same or equivalent function to the original. In accordance with an exemplary embodiment, for example, an arbitrary structure or structures may be added to the aforementioned configuration.

While the puncture needle-side expanding section is formed over the total length in the longitudinal direction of the puncture needle in this embodiment, this is not restrictive. For example, the puncture needle-side expanding section may be formed up to an intermediate part in the longitudinal direction of the puncture needle, namely, be formed over part of the longitudinal range of the puncture needle.

In the medical device described above, both the puncture needle and the sheath have the rotation restraining section for restraining rotation of the sheath, but this configuration is not restrictive. For example, a configuration may be adopted in which one of the puncture needle and the sheath has the rotation restraining section.

While both the puncture needle and the sheath are flat shaped in cross section in the aforementioned medical device, this is not restrictive. For example, a configuration may be adopted in which the puncture needle is flat shaped in cross section, whereas the sheath is, in the second state, formed into a flat shape conforming to the cross-sectional shape of the puncture needle. Alternatively, only the sheath may be flat shaped in cross section.

While the sheath is passed through the primary insertion hole while coming into the second state, this is not restrictive. For example, the sheath may be passed through the primary insertion hole after the transition to the second state is completed.

The medical device may have a configuration in which, for example, the vaginal-insertion member is omitted and the restraining means restrains only the positional relation of the puncture needle (puncture member) and the urethral-insertion member.

While the puncture needle of the puncture member is curved in a circular arc shape in its entirety, this is not restrictive. For example, the puncture needle may have a circular arc-shaped curved section, only at part or portion of the puncture needle. In accordance with an exemplary embodiment, for example, it can be sufficient for the puncture needle to have a circular arc-shaped curved section at least at part of the puncture needle.

It is sufficient for the puncture needle of the puncture member to have a curved section at least at part of the puncture member. For instance, the puncture needle may be curved in an elliptic arc shape in its entirety, or may have an elliptic arc-shaped curved section only at part of the puncture needle. Thus, the puncture needle may have an elliptic arc-shaped section at least at part of the puncture needle.

While description has been made of a case where the medical device is applied to a device for use in embedding in a living body an embeddable implant for treatment of female urinary incontinence, the use of the medical device is not limited to the described one.

For example, the target of the application of the present disclosure can include excretory disorders attendant on the weakening of the pelvic floor muscle group (urinary urgency, frequent urination, urinary incontinence, fecal incontinence, urinary retention, dysuria or the like), and pelvic floor disorders including pelvic organ prolapse, vesicovaginal fistula, urethrovaginal fistula, pelvic pain or the like. In the pelvic organ prolapse, there are included disorders of cystocele, enterocele, rectocele, hysterocele and the like. Alternatively, there are included such disorders as anterior vaginal prolapse, posterior vaginal prolapse, vaginal stump prolapse, vaginal vault prolapse and the like in which the naming method thereof is based on the prolapsed vaginal-wall part.

Also, overactive tissues include bladder, vagina, uterus, bowel and the like. Less-active tissues include bones, muscles, fascias, ligaments and the like. In particular, in the case of pelvic floor disorders, the less-active tissues include an obturator fascia, a coccygeus fascia, a cardinal ligament, an uterosacral ligament, a sacrospinous ligament and the like.

For the procedure for interlocking an overactive tissue in the pelvic floor disorder with the less-active tissue, there are included a retropubic sling surgery, a transobturator sling surgery, transobturator tape (TOT), a tension-free vaginal mesh (TVM) surgery, a uterosacral ligament suspension (USLS) surgery, a sacrospinous ligament fixation (SSLF) surgery, an iliococcygeus fascia fixation surgery, a coccygeus fascia fixation surgery, and the like.

The medical device of the present disclosure is a medical device to be used when indwelling a belt-shaped flexible implant into a living body. The medical device can include an elongated puncture needle which, by piercing the living body, forms a primary insertion hole in the living body; and a sheath into and out of which the puncture needle can be inserted and removed and which, by passing through the primary insertion hole in an assembled state with the puncture needle inserted therein, makes the primary insertion hole into a secondary hole so as to permit the puncture needle to be inserted in and passed through the secondary hole, wherein the puncture needle has a puncture needle-side expanding section which, when forming the primary insertion hole, expands the primary insertion hole to the same degree as the width of the implant. Therefore, an insertion hole having a required minimum size for stable indwelling of the implant therein can be formed easily and reliably.

Accordingly, the medical device of the present disclosure has industrial applicability.

The detailed description above describes medical device. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical device for indwelling a belt-shaped flexible implant into a living body, the medical device comprising:
an elongated puncture needle which, by piercing the living body, forms a primary insertion hole in the living body; and
a sheath into and out of which the puncture needle can be inserted and removed and which, by passing through the primary insertion hole in an assembled state with the puncture needle inserted therein, makes the primary insertion hole into a secondary insertion hole so as to permit the implant to be inserted in and passed through the secondary insertion hole,
wherein the puncture needle has a puncture needle-side expanding section which, when forming the primary insertion hole, expands the primary insertion hole to a same degree as a width of the implant.

2. The medical device according to claim 1,
wherein the puncture needle is formed, at least at part in a longitudinal direction of the puncture needle, with a flat section which is flat shaped in cross section, and the flat section functions as the puncture needle-side expanding section.

3. The medical device according to claim 1,
wherein the sheath has a sheath-side expanding section which, when forming the secondary insertion hole, causes the secondary insertion hole to retain an expanded state of the primary insertion hole expanded by the puncture needle-side expanding section.

4. The medical device according to claim 1,
wherein at least one of the puncture needle and the sheath has a rotation restraining section which restrains rotation of the sheath relative to the puncture in the assembled state.

5. The medical device according to claim 4,
each of the puncture needle and the sheath is formed, at least at part in a longitudinal direction thereof, with a flat section which is flat shaped in cross section, and the flat sections, by overlapping with each other in the assembled state, function as the rotation restraining section.

6. The medical device according to claim 1,
wherein an overall length of the sheath is greater than an overall length of the puncture needle.

7. The medical device according to claim 1,
wherein the puncture needle has a curved shape by being curved in a circular arc shape.

8. The medical device according to claim 7,
wherein the sheath is flexible and is, in the assembled state, deformed so as to be along the curved shape.

9. The medical device according to claim 7,
wherein the sheath is preliminarily re-shaped so as to be along the curved shape in the assembled state.

10. The medical device according to claim 1, further comprising
an elongated insertion section to be inserted into a biological lumen,
wherein the sheath is formed, at least at part in a longitudinal direction of the sheath, with a flat section which is flat shaped in cross section, and the flat section is configured, when the sheath forms the secondary insertion hole, to be disposed in parallel to the insertion section.

11. The medical device according to claim 1, wherein the medical device is configured to be used for treating a disorder in a pelvic organ by indwelling the implant between a urethral lumen and a vaginal cavity.

12. The medical device according to claim 1, comprising:
the implant; and
an implant package having a wrapping material, the wrapping material including a bag-shaped wrapping material main body in which to accommodate the implant, and a flexible linear body for pulling the wrapping material main body.

13. The medical device according to claim 1,
wherein the implant has a stopper which makes contact with a body surface when the implant is indwelled in the living body.

14. A medical device for indwelling a belt-shaped flexible implant into a living body, the medical device comprising:
a puncture needle which, by piercing the living body, forms a primary insertion hole in the living body; and
a sheath into and out of which the puncture needle can be inserted and removed and which, by passing through the primary insertion hole in an assembled state with the puncture needle inserted therein, makes the primary insertion hole into a secondary insertion hole so as to permit the implant to be inserted in and passed through the secondary insertion hole,
wherein the sheath has a sheath-side expanding section which, when forming the secondary insertion hole, expands the secondary insertion hole to a same degree as a width of the implant.

15. The medical device according to claim 14,
wherein the sheath is formed, at least at part in a longitudinal direction of the sheath, with a flat section which is flat shaped in cross section, and the flat section functions as the sheath-side expanding section.

16. A method of indwelling a belt-shaped flexible implant into a living body, the method comprising:
inserting a medical device into the living body, the medical device including an elongated puncture needle which, by piercing the living body, forms a primary insertion hole in the living body, and a sheath into and out of which the puncture needle can be inserted and removed;
passing the medical device through the primary insertion hole in an assembled state with the puncture needle inserted in the sheath;
making the primary insertion hole into a secondary insertion hole so as to permit the implant to be inserted in and passed through the secondary insertion hole; and
wherein the puncture needle has a puncture needle-side expanding section which, when forming the primary insertion hole, expands the primary insertion hole to a same degree as a width of the implant.

17. The method according to claim 16,
wherein the puncture needle is formed, at least at part in a longitudinal direction of the puncture needle, with a flat section which is flat shaped in cross section, and the flat section functions as the puncture needle-side expanding section.

18. The method according to claim 16,
wherein the sheath has a sheath-side expanding section which, when forming the secondary insertion hole, causes the secondary insertion hole to retain an expanded state of the primary insertion hole expanded by the puncture needle-side expanding section.

19. The method according to claim 16,
wherein at least one of the puncture needle and the sheath has a rotation restraining section which restrains rotation of the sheath relative to the puncture in the assembled state; and
each of the puncture needle and the sheath is formed, at least at part in a longitudinal direction thereof, with a flat section which is flat shaped in cross section, and the flat sections, by overlapping with each other in the assembled state, function as the rotation restraining section.

20. The method according to claim 16, comprising:
an elongated insertion section to be inserted into a biological lumen,
wherein the sheath is formed, at least at part in a longitudinal direction of the sheath, with a flat section which is flat shaped in cross section, and the flat section is configured, when the sheath forms the secondary insertion hole, to be disposed in parallel to the insertion section.
